(19) United States
(12) Patent Application Publication
(10) Pub. No.: US 2023/0240572 A1
(43) Pub. Date: Aug. 3, 2023

(54) PSYCHOLOGICAL STRESS CALCULATION DEVICE, PSYCHOLOGICAL STRESS CALCULATION METHOD, AND PROGRAM

(71) Applicant: NIPPON TELEGRAPH AND TELEPHONE CORPORATION, Tokyo (JP)

(72) Inventors: Mana SASAGAWA, Musashino-shi, Tokyo (JP); Daiki SATO, Musashino-shi, Tokyo (JP); Takashi ISEZAKI, Musashino-shi, Tokyo (JP); Tomoki WATANABE, Musashino-shi, Tokyo (JP)

(73) Assignee: NIPPON TELEGRAPH AND TELEPHONE CORPORATION, Tokyo (JP)

(21) Appl. No.: 18/013,175

(22) PCT Filed: Jul. 3, 2020
(86) PCT No.: PCT/JP2020/026197

§ 371 Date: Dec. 27, 2022

(51) Int. Cl. A61B 5/16; A61B 5/00; G16H 50/20

(52) U.S. Cl. CPC A61B 5/16; A61B 5/72; G16H 50/20

(57) ABSTRACT

A psychological oppression feeling that is given to a person by a robot is accurately calculated.

There is provided a psychological oppression feeling calculating device including: a first acquisition unit (S02) that acquires a relative positional relationship between a person and a plurality of robots including the number of the plurality of robots with respect to the person, a moving speed of the robot, and a distance between the person and the robot; a second acquisition unit (S01) that acquires a density of the robots with respect to the person; and a calculation unit (S03 and S04) that calculates a psychological oppression feeling given to the person by the robots from results acquired by the first acquisition unit and the second acquisition unit.

### TECHNICAL FIELD

The present invention relates to a psychological oppression feeling calculating device, a psychological oppression feeling calculating method, and a program.

### BACKGROUND ART

As research on robots for coexistence with a person, there have been attempts to examine a psychological oppression feeling (a degree of disturbance) given to a person by a robot. As parameters required for quantifying the psychological oppression feeling, a technique using the number of robots (Non Patent Literature 1), a technique using a speed of a robot (Non Patent Literature 2), and a technique using a distance between a robot and a person (Non Patent Literature 3) are considered.

For example, in the technique described in Non Patent Literature 1, a physiological and psychological response according to the number of small robots in autonomous travel is measured. In addition, in the technique described in Non Patent Literature 2, compensatory behavior according to a speed of a robot approaching a person is observed. Further, in the technique described in Non Patent Literature 3, a distance at which a person no longer wants to be approached by one small robot in autonomous travel is measured for each angle and speed.

### CITATION LIST

### Non Patent Literature

Non Patent Literature 1: Podevijn, G., O'grady, R., Mathews, N., Gilles, A., Fantini-Hauwel, C., & Dorigo, M. (2016). Investigating the effect of increasing robot group sizes on the human psychophysiological state in the context of human-swarm interaction. Swarm Intelligence, 10(3), 193-210.

Non Patent Literature 2: Aziez Sardar, Michiel Joosse, Astrid Weiss, and Vanessa Evers. 2012. Don't stand so close to me: users' attitudinal and behavioral responses to personal space invasion by robots. In Proceedings of the seventh annual ACM/IEEE international conference on Human-Robot Interaction (HRI '12). Association for Computing Machinery, New York, N.Y., USA, 229-230.

Non Patent Literature 3: Miyu Aoki & Akiko Watanabe. (2011). A STUDY ON THE DISTANCES OF AN UPRIGHT/CHAIR-SITTING SMALL MOBILE ROBOT TO MALE ADULT INDIVIDUALS. Journal of Architecture and Planning by The Architectural Institute of Japan, 76(664), 1093-1100.

### SUMMARY OF INVENTION

### Technical Problem

In a case where a psychological oppression feeling given to a person by a plurality of robots is calculated as a numerical value, it is considered that an insufficient value is obtained from only the above-described various parameters which are currently considered, that is, the number of the robots, a speed of the robot, and a distance between the person and the robot.

The present invention has been made in view of the above circumstances, and an object of the present invention is to provide a psychological oppression feeling calculating device, a psychological oppression feeling calculating method, and a program capable of accurately calculating a psychological oppression feeling given to a person by a robot.

### Solution to Problem

An aspect of the present invention includes: a first acquisition unit that acquires a relative positional relationship between a person and a plurality of robots including the number of the plurality of robots with respect to the person, a moving speed of the robot, and a distance between the person and the robot; a second acquisition unit that acquires a density of the robots with respect to the person; and a calculation unit that calculates a psychological oppression feeling given to the person by the robots from results acquired by the first acquisition unit and the second acquisition unit.

### Advantageous Effects of Invention

According to an aspect of the present invention, it is possible to accurately calculate a psychological oppression feeling given to a person by a robot.

### BRIEF DESCRIPTION OF DRAWINGS

FIG. 1 is a diagram illustrating a configuration of an entire experimental system for measuring a psychological oppression feeling according to an embodiment of the present invention.

FIG. 2 is a flowchart illustrating a series of processing contents executed by a personal computer according to the embodiment.

FIG. 3 is a diagram schematically illustrating images of robots on a desk that are obtained by a main surveillance camera and a user camera according to the embodiment.

FIG. 4 is a diagram schematically illustrating images of robots on a desk that are obtained by the main surveillance camera and the user camera according to the embodiment.

### DESCRIPTION OF EMBODIMENTS

Hereinafter, an embodiment in a case where the present invention is applied to a measurement system for a psychological oppression feeling from robots will be described.

### Configuration

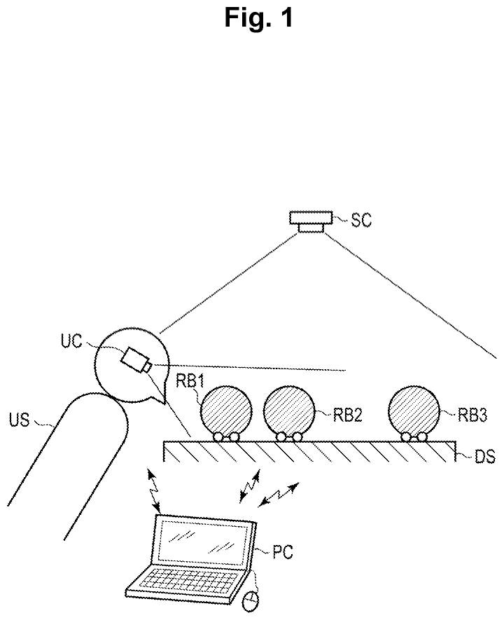

FIG. 1 is a diagram illustrating a configuration of an entire experimental environment of a measurement system according to the present embodiment. In FIG. 1, it is assumed that a user US who is a person observes a state where a plurality of robots, for example, three robots RB1 to RB3 randomly move on a desk DS. The entire periphery including the user US and the robots RB1 to RB3 on the desk DS is captured by, for example, a main surveillance camera SC provided on a ceiling.

On the other hand, a user camera UC is also attached to a portion of a body of the user US, for example, a head. The camera UC captures an image of a scene that includes the robots RB1 to RB3 on the desk DS and is viewed by the user US.

The attachment position of the user camera UC is desirably closer to a position of an eye of the user US because of the necessity to reduce parallax. For example, as illustrated in FIG. 1, it is considered that the user camera UC is attached to one temporal region by using a headband in combination.

Both an image signal obtained by image capturing of the main surveillance camera SC and an image signal obtained by image capturing of the camera UC are wirelessly transmitted to a personal computer PC in the vicinity of the desk DS and the user US.

In the personal computer PC, an application program for executing a series of processing of the measurement system performed in the present embodiment is installed in advance. The personal computer PC acquires image signals from the main surveillance camera SC and the camera UC by, for example, a wireless LAN technique based on an IEEE 802.11a/11b/11g/11n standard or a wireless communication technique based on a Bluetooth (registered trademark) standard, and also controls movement of a body of each of the robots RB1 to RB3.

The robots RB1 to RB3 move on the desk DS without interfering with each other by autonomous travel, and execute movement corresponding to a position of the user US according to a control instruction from the personal computer PC.

### Operation

Hereinafter, an operation in the case of measuring a psychological oppression feeling of the user US with respect to the robots RB1 to RB3 by the application program installed in the personal computer PC will be described.

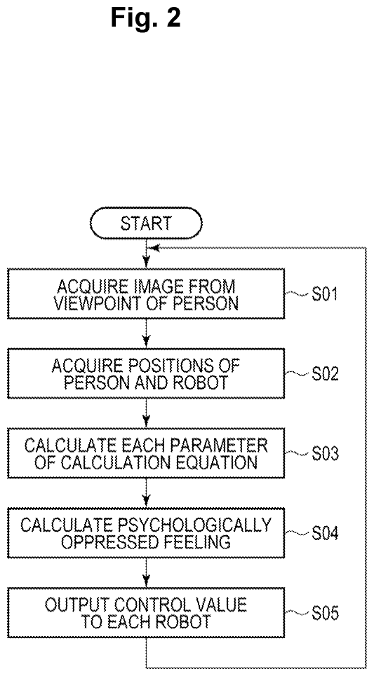

FIG. 2 is a flowchart illustrating a series of processing contents executed by the application program on the personal computer PC.

At the beginning of processing, the personal computer PC acquires, from the user camera UC, an image including the robots RB1 to RB3 from a viewpoint of the person (step S01).

Next, the personal computer PC acquires an image from the main surveillance camera SC, as an image for acquiring a relative positional relationship between the user US who is a person and the robots RB1 to RB3 (step S02).

The personal computer PC calculates various parameters indicating a relative positional relationship between the user US and the robots RB1 to RB3 by performing contour emphasis processing on the image obtained by the main surveillance camera SC, recognizing and separating subjects (the user US and the robots RB1 to RB3) in the image, and obtaining an imaging view angle and a focus length of the main surveillance camera SC, a distance to the desk DS, and a position and a size of each subject in the image (step S03).

Examples of the parameters to be calculated include the number N of the robots, moving speeds Sᵢ (=S₁ to S₃) of the robots (RB1 to RB3), and distances Kᵢ (=K₁ to K₃) between the robots and the person. The moving speeds of the robots are calculated from displacements in positions of the robots RB1 to RB3 with respect to a plurality of previous images acquired on the order of milliseconds in a case where a frame frequency at which the main surveillance camera SC performs image capturing is set to, for example, approximately 1000 [frames/second].

In addition, a density D is calculated as a parameter based on the image from the user camera UC attached to the user US. The density D indicates a ratio (%) of an area of the robots RB1 to RB3 in the image obtained by the user camera UC.

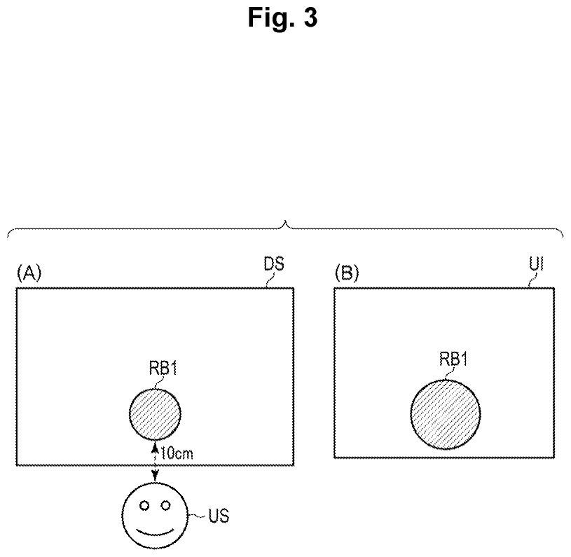

FIG. 3 is a diagram schematically illustrating an image (FIG. 3(A)) obtained by capturing the desk DS on which one robot RB1 is placed by the main surveillance camera SC from above and an image UI (FIG. 3(B)) obtained by capturing the robot RB1 on the desk DS by the user camera UC in the same state.

As illustrated in FIG. 3(A), the distance (K₁) between the one robot RB1 and the user US is 10 [cm]. At that time, as illustrated in FIG. 3(B), a ratio of an area of the robot RB1 in the image UI obtained by the user camera UC is, for example, 5 [%].

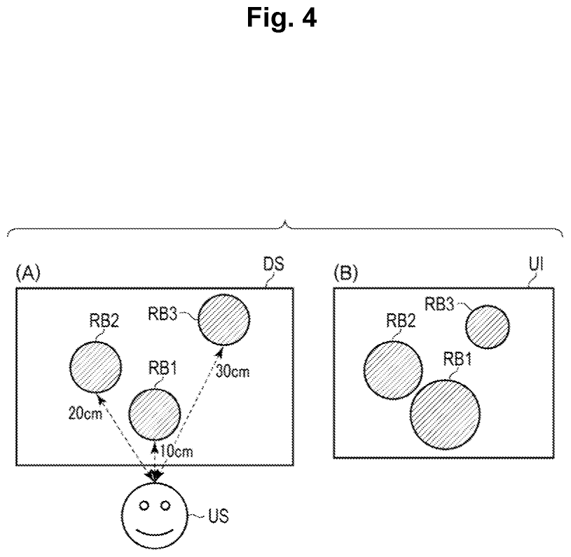

FIG. 4 is a diagram schematically illustrating an image (FIG. 4(A)) obtained by capturing the desk DS on which three robots RB1 to RB3 are placed by the main surveillance camera SC from above and an image UI (FIG. 4(B)) obtained by capturing the robots RB1 to RB3 on the desk DS by the user camera UC in the same state.

As illustrated in FIG. 4(A), distances (K₁ to K₃) between the three robots RB1 to RB3 and the user US are respectively 10 [cm], 20 [cm], and 30 [cm]. At that time, as illustrated in FIG. 4(B), a ratio of an area of the robots RB1 to RB3 in the image UI obtained by the user camera UC is, for example, 20 [%].

For the parameters including the number N of the robots, the moving speeds Sᵢ (=S₁ to S₃) of the robots (RB1 to RB3), the distances Kᵢ (=K₁ to K₃) between the robots and the person, and the density D, it is necessary to adjust coefficients and offsets as appropriate.

The personal computer PC calculates a psychological oppression feeling P, which is given to the user US by the presence of the robots RB 1 to RB3 moving on the desk DS, using various parameters (step S04).

Hereinafter, calculation of the psychological oppression feeling P will be described.

First, setting of an equation part that is calculated for each robot will be described. In each of the plurality of N robots, it is considered that the psychological oppression feeling P increases as the moving speeds Sᵢ (i=1, 2, . . . , N) increase and as the distances Kᵢ (i=1, 2, . . . , N) between the robots and the person decrease. Therefore, an equation is set up such that the psychological oppression feeling P increases in proportion to the moving speeds Sᵢ and in inverse proportion to the distances Kᵢ. For example, it is assumed that the psychological oppression feeling P given by an i-th robot RBi is represented as “Sᵢ/Kᵢ”.

Next, setting of an equation part that is calculated in consideration of all the N robots will be considered. Since it is considered that the psychological oppression feeling P increases as the number N of the robots increases, by adding the parts calculated for each of the psychological oppression feeling P described above, an equation is set such that the psychological oppression feeling P increases in proportion to the number N of the robots. In addition, since it is considered that the psychological oppression feeling P increases as the density D of the robots increases, an equation is set such that the psychological oppression feeling P increases in proportion to the density D. From the above points, an equation of the psychological oppression feeling P is set as follows using all the parameters (N, S, K, D). That is, Equation 1 is obtained.

$$P = D {\sum }_{i = 1}^{N} \frac{{S}_{i}}{{K}_{i}} \qquad [ Math . 1 ]$$

In a case where the calculation equation is actually used, it is necessary to adjust a coefficient or an offset of each parameter and an allowable numerical value range according to a situation. Specifically, assuming that the number N of the robots is 0 to 5, the moving speed S is 0 to 20 [cm/sec], the distance K between the robot and the person is 0 to 75 [cm], and the density D is 0 to 100 [%], a method of adjusting the equation in a case of calculating the psychological oppression feeling P as a numerical value between 0 and 1 will be described.

In the case of N=0, since there is no robot in front of the user US, D=0, and as a result, the psychological oppression feeling P=0.

As illustrated in FIG. 3, in the case of N=1, in order to calculate the psychological oppression feeling P as a numerical value between 0 and 1, it is necessary to set a numerical value of D to 1/100, set both the moving speed S and a reciprocal 1/K of the distance K to be equal to or smaller than 1, and further divide S and 1/K by the number N of the robots. Therefore, assuming that actually acquired values are d, sᵢ, and kᵢ, for example, d, sᵢ, and kᵢ are calculated as follows.

D=d/100,

Sᵢ=(1+sᵢ)/(1+S_{MAX}),

Kᵢ=1+kᵢ

From the above, the equation in a case where it is desired to calculate the psychological oppression feeling P as a numerical value between 0 and 1 is adjusted as follows. That is, Equation 1 is obtained.

[Math. 2]

$$When N \geq 1 \\ P = \frac{d}{100} \frac{1}{N} {\sum }_{i = 1}^{N} \frac{( 1 + {s}_{i} )}{( 1 + {S}_{MAX} )} \frac{1}{( 1 + {k}_{i} )} \\ When N = 0 \\ P = 0$$

A specific calculation method of the psychological oppression feeling P using the calculation equation will be described.

As illustrated in FIG. 3, in a case where N=1, d=5 [%], s₁=5 [cm/sec], and k₁=10 [cm], the psychological oppression feeling P is represented as follows.

$$\begin{matrix}P = \frac{5}{100} \frac{1}{1} {\sum }_{n = 1}^{1} \frac{( 1 + 5 )}{( 1 + 20 )} \frac{1}{( 1 + 10 )} \\ = 0.0013\end{matrix} \qquad [ Math . 3 ]$$

Further, as illustrated in FIG. 4, in a case where N=3, d=20 [%], s₁=5 [cm/sec], s₂=10 [cm/sec], s₃=15 [cm/sec], k₁=10 [cm], k₂=20 [cm], and k₃=30 [cm], the psychological oppression feeling P is represented as follows.

$$\begin{matrix}P = \frac{20}{100} \frac{1}{3} {\sum }_{i = 1}^{3} \frac{( 1 + {s}_{i} )}{( 1 + 20 )} \frac{1}{( 1 + {k}_{i} )} \\ = \frac{1}{15} [ \frac{( 1 + 5 )}{( 1 + 20 )} \frac{1}{( 1 + 10 )} + \frac{( 1 + 10 )}{( 1 + 20 )} \frac{1}{( 1 + 20 )} + \frac{( 1 + 15 )}{( 1 + 20 )} \frac{1}{( 1 + 30 )} ] \\ = 0.005\end{matrix} \qquad [ Math . 4 ]$$

Next, a method of acquiring a specific value of each parameter will be described.

As described above, the number N of the robots, the moving speeds S of the robots, and the distances K between the robots and the person are calculated from the positions of the user US and the robots RB1 to RB3 in the plurality of images continuously acquired on the order of milliseconds and the displacement information of the robots RB1 to RB3 moving between the plurality of images in a case where the frame frequency at which the main surveillance camera SC captures an image is, for example, approximately 300 [frames/second].

Further, the density D is obtained by calculating the area of the robots RB1 to RB3 in the image obtained by capturing the desk DS by the user camera UC attached to the head or the like of the user US.

After calculating the psychological oppression feeling P in this way in step S04, the personal computer PC transmits, to each of the robots RB1 to RB3, a control signal for moving each robot body based on control contents corresponding to the psychological oppression feeling P which is preset (step S05). The personal computer PC ends a series of processing operations described above, and returns to processing from step S01 to continue the operations.

As specific control contents by the personal computer PC, a control for increasing the psychological oppression feeling P, in a case where the psychological oppression feeling P is lower than a certain threshold value, by instructing the robot to move in a movement direction toward the user US and at a moving speed in order from the robot RBi of which the distance Ki from the user US is farthest, or conversely, a control for decreasing the psychological oppression feeling P, in a case where the psychological oppression feeling P is higher than a certain threshold value, by instructing the robot to move in a movement direction away from the user US and at a moving speed in order from the robot RBi of which the distance Ki from the user US is closest may be considered.

As described above, by the personal computer PC repeatedly executing processing of step S01 to step S05, the user US who observes the robots RB1 to RB3 moving on the desk DS can control the psychological oppression feeling P from the robots RB1 to RB3 within an appropriate range.

It is also considered that, for example, a size, a height, a color, and a shape of the robot, a type and a volume of a sound emitted by the robot, a position of the robot in a field of view of the person, and the like are taken into account in addition to types of the parameters described above.

Regarding the position of the robot in the field of view of the person, specifically, it is determined whether the robot is present in the central field of view of the person, in the peripheral field of view of the person, or outside the field of view of the person. Specifically, for example, in a case where the robot is located at 0° to ±30° from a forward direction of the person, it is determined that the robot is located in the central field of view of the person, in a case where the robot is located at ±30° to ±100° from the forward direction of the person, it is determined that the robot is located in the peripheral field of view of the person, and otherwise, it is determined that the robot is outside the field of view of the person.

It is considered that the psychological oppression feeling P increases as the robot is present in front of the person. Therefore, it is possible to more accurately calculate the psychological oppression feeling P by setting a coefficient according to a dispersion degree of the positions of the robots from the forward direction and using the coefficient for calculation of the density D such that the psychological oppression feeling P increases as the positions are closer to 0° from the forward direction of the person.

In the above-described embodiment, a ratio of the area of the robots RB1 to RB3 in the image captured by the user camera UC attached to the head of the user US is used as the density D. On the other hand, as a method of calculating the density D in a pseudo manner, for example, the density D may be calculated in consideration of only the positions of the robots RB1 to RB3.

Specifically, for example, the robots are divided into a plurality of groups according to the positions of the robots obtained by the main surveillance camera SC, by using k-means clustering or the like which is one of non-hierarchical clustering methods. Next, a dispersion degree of the robots for each group is calculated. It is considered to use a sum of reciprocals of the dispersion degrees obtained in this way as the density D.

Further, the dispersion degree of the robots for each group may be obtained by multiplying a coefficient which is set in consideration of the distance to the user who is a person, that is, by multiplying by a coefficient having a higher value as the group is closer to the person and has a lower dispersion degree.

As described above, in particular, when the total number of the robots is large, in a case where the density D can be calculated by a clustering method using the position of each robot and the relative distance between the robot and the person as necessary, it is not necessary to attach a camera to a head or the like of the person, and it is possible to calculate the psychological oppression feeling P with a simpler system configuration.

In the present embodiment, processing in a case where overall control is performed by the application program installed in the personal computer PC has been described. On the other hand, the present invention is not limited thereto. For example, assuming that each of the plurality of robots is configured to recognize a distance or a positional relationship between another robot or the person and some or all of the robots are configured to calculate the psychological oppression feeling P given to the person, a control operation according to the calculation result may be executed.

### Advantageous Effects of the Embodiment

As described above, according to the present embodiment, it is possible to accurately calculate a psychological oppression feeling given to a person by a robot.

Further, in the present embodiment, the density D is calculated by the ratio of the area of the robots RB1 to RB3 in the image in the field of view of the user US that is captured by the user camera UC attached to the head or the like of the user US who is a person. Therefore, it is possible to directly acquire the subjective density D felt by the person by a relatively simple calculation method.

A case where the device according to the present invention is realized by the application program installed in the personal computer PC illustrated in FIG. 1 has been described. On the other hand, the program may be provided by being recorded in a recording medium or via a network.

Note that the present invention is not limited to the above-described embodiment, and various modifications may be made in the implementation stage without departing from the spirit of the inventions. Further, the above-described embodiment includes inventions in various stages, and various inventions can be extracted by an appropriate combination from a plurality of disclosed components. For example, even though some components are deleted from all the components described in the embodiment, in a case where the problems described in Technical Problem can be solved and the effects described in Advantageous Effects of Invention can be obtained, a configuration from which the components are deleted can be extracted as an invention.

### REFERENCE SIGNS LIST

DS desk
PC personal computer
RB1 to RB3 robot
SC main surveillance camera
UC user camera
US user (person)

1. A psychological oppression feeling calculating device comprising:
a processor; and
a storage medium having computer program instructions stored thereon, when executed by the processor, perform to:
acquires a relative positional relationship between a person and a plurality of robots including the number of the plurality of robots with respect to the person, a moving speed of the robot, and a distance between the person and the robot;
acquires a density of the robots with respect to the person; and
calculates a psychological oppression feeling given to the person by the robots from results acquired by the first acquisition unit and the second acquisition unit.

2. The psychological oppression feeling calculating device according to claim 1,
wherein the density of the robots with respect to the person includes a ratio of an area of the robots in a field of view of the person.

3. The psychological oppression feeling calculating device according to claim 1,
wherein the density of the robots with respect to the person includes a dispersion degree of the robots from a center in a forward direction of the person.

4. The psychological oppression feeling calculating device according to claim 1,
wherein the density of the robots with respect to the person includes a dispersion degree for each group obtained by grouping positions of the robots with respect to the person.

5. A psychological oppression feeling calculating method comprising:
a first acquisition step of acquiring a relative positional relationship between a person and a plurality of robots including the number of the plurality of robots with respect to the person, a moving speed of the robot, and a distance between the person and the robot;
a second acquisition step of acquiring a density of the robots with respect to the person; and
a calculation step of calculating a psychological oppression feeling given to the person by the robots from results acquired by the first acquisition step and the second acquisition step.

6. A non-transitory computer-readable medium having computer-executable instructions that, upon execution of the instructions by a processor of a computer, cause the computer to function as the psychological oppression feeling calculating device according to claim 1.
